(19) United States
(12) Patent Application Publication
(10) Pub. No.: US 2019/0216031 A1
(43) Pub. Date: Jul. 18, 2019

(54) Algae Culturing

(71) Applicant: Christopher James Hintz, Savannah, GA (US)

(72) Inventor: Christopher James Hintz, Savannah, GA (US)

(21) Appl. No.: 16/248,817

(22) Filed: Jan. 16, 2019

Related U.S. Application Data
(60) Provisional application No. 62/618,105, filed on Jan. 17, 2018.

(51) Int. Cl. A01G 33/00; A01K 61/10; C12N 1/12; A23K 50/80; A01K 61/50

(52) U.S. Cl. CPC A01G 33/00; A01K 61/10; A01K 61/50; A23K 50/80; C12N 1/12

(57) ABSTRACT

The present embodiments relate to selectively growing microalgae of a size suitable for the culturing of planktivorous finfish such as sardines, menhaden, herring, anchovy or shellfish such as oysters, clams, mussels, scallops, and sessile invertebrates such as sponges, coral, sea jellies, and other Cnidaria, or sedentary polychaete or bristle worms. A nitrogen to phosphorous content ratio of a nutrient medium may be measured. Nitrogen and/or phosphorous may be added to adjust the nitrogen to phosphorous content ratio in the nutrient medium to a ratio of 100:1 or higher. Microalgae may be grown in the adjusted nutrient medium. The environment may be controlled to produce microalgae with a predetermined diameter.

### CROSS-REFERENCE TO RELATED APPLICATIONS

This application claims the benefit of U.S. Provisional

Application No. 62618105, filed 17 Jan. 2018, which is hereby incorporated by reference in its entirety.

### BRIEF DESCRIPTION OF THE SEVERAL VIEWS OF THE DRAWINGS

Examples of several of the various embodiments of the present invention are described herein with reference to the drawings.

FIG. 1 is an example process flow diagram as per an aspect of an embodiment of the present invention.

FIG. 2 is an example process flow diagram as per an aspect of an embodiment of the present invention.

FIG. 3 is an example process flow diagram as per an aspect of an embodiment of the present invention.

FIG. 4 is an example diagram of a culturing apparatus as per an aspect of an embodiment of the present invention.

FIG. 5 is an example diagram of a culturing apparatus as per an aspect of an embodiment of the present invention.

FIG. 6 is an example diagram of a culturing apparatus as per an aspect of an embodiment of the present invention.

### DETAILED DESCRIPTION OF EMBODIMENTS

Algae culture is an aquatic system, marine or freshwater, to facilitate and support the reproduction of monospecific or polyspecific microalgaes or meioalgaes. This system may have a dedicated continuous harvesting of cultured organisms, or may be operated in batches. A marine aquatic system may be comprised of salt or brackish water as a culture medium, an aquatic mixture with dissolved chemical constituents that can support biological growth. A freshwater aquatic system is essentially free of salt. The culturing medium may have its chemical composition altered to support optimized growth of organisms living in the culture medium. Environmental parameters of this culture medium typically important to aquatic algae culture include temperature, culturing medium salinity, illuminating light intensity, illumination light spectrum including peak wavelength and continuous or non-continuous (emission) spectral characteristics across the visible light wave lengths of 400-700 nanometers (nm).

Recently hatched planktivorous organisms such as oyster spat are unable to feed on microalgae above a certain physical size. The normal method of feeding planktivorous organisms until they grow of sufficient size to filter feed on large microalgae is to utilize naturally occurring marine or fresh water containing microalgae or, alternatively, to culture algae in the culturing facility without differentiating among the size of the algae.

Natural waters are those waters found in the environment, either considered pristine or potentially polluted, but otherwise unaltered before being collected into local aquaculture facilities.

Natural nutrients may be from any body of water (marine or fresh) found in the environment that has the appropriate nitrogen to phosphorus ratio, N:P, and does not need modification. This nutrient source may be considered eutrophic and have been influenced by anthropogenic sources, but serves as an appropriate nutrient source for the target algae species. The nitrogen to phosphorus ratio, N:P, is a molar ratio of the elements nitrogen and phosphorus found in water. This ratio may be typically measured using the inorganic forms of available nitrogen, ammonia and nitrate, to inorganic forms of phosphorus, phosphate. In some cases, dissolved organic compounds containing nitrogen and phosphorus may also be included in this N:P ratio, although the organic concentrations are typically low and their inclusion does not usually alter the overall N:P significantly.

Artificial nutrients may be from the effluent of an industrial or municipal wastewater treatment plant (IWTP or MWTP)) or may be created from natural nutrient source and supplemented by the addition of chemicals (e.g., fertilizer) or by other nutrient addition (e.g., manure) that supports a phosphorus-limited culture media. Effluent is liquid waste or sewage discharged into another body of water such as natural or man-made pond, lake, stream, river or ocean. Effluent may also be referred to as waste, sewage, wastewater, effluvium, outflow, discharge, or emission. Effluent, in engineering, is the stream exiting a chemical reactor.

Wastewater nitrogen concentration ranges widely from 1-5 mg per liter up to greater than 100 mg per liter of effluent as inorganic nitrogen ions nitrate and ammonia. Phosphorous in effluent also widely ranges from less than 1 mg per liter to greater than 10 mg per liter effluent as the inorganic phosphorus ion phosphate.

When natural waters or the culture medium does not meet the necessary nutrient concentrations to support photosynthetic growth with the available light, these waters are considered nutrient limited. A nutrient medium may be comprised of necessary nutrients that are not sufficiently available in the natural water or culture medium including but not necessarily the macronutrients, nitrogen, phosphorus, silica, or micronutrients, iron, copper, molybdenum, zinc, manganese, or vitamins such as biotin, thiamine may be added to supplement the culture medium and ultimately support the continued biomass growth.

The mineral nutrient requirements for algae include sources of the following ions with suitable ranges of concentrations of ions as follows:

| | |
|---|---|
| Magnesium (Mg²⁺) | 0.001 to 0.05M. |
| Potassium (K⁺) | 001 to 0.1M. |
| Iron (Fe²⁺ or Fe³⁺) | 0.1 × 10⁻⁵ to 0.5 × 10⁻⁴M. |
| Sulphate (SO₄²⁻) | 0.005 to 0.1M. |
| Phosphate (PO₄³⁻) | 0.001 to 0.05M. |
| Fixed nitrogen | 0.0015 to 0.1M. |

High-phosphorus media found in some industrial wastewater and occasionally naturally occurring in regions near phosphorite deposits is defined as a nitrogen to phosphorus ratio (N:P) less than 30:1 N:P with sufficient nutrients to support algal growth in the available light. The high-phosphorus nutrient regime below 30:1 N:P promotes cyanobacteria and blue-green algae that are not preferred genotypes for this application and thus should be avoided. An algae genotype is a particular genetic characteristic within a group of organisms. In this case the preferred genotypes to culture are traditional single-cell photosynthetic algaes (primarily chlorophyllic green algaes, but less common brown or red algaes) that have a nominal cell size less than 5 micrometers.

Phosphorus replete is defined as an environment or growth environment containing high enough phosphorus concentration to support photosynthetic growth in the available light. Similarly, nitrogen replete is defined as a growth or culture environment containing high enough nitrogen concentration to support photosynthetic growth in the available light. Nutrient replete is growth or culture environment containing high enough concentrations of all nutrients to support photosynthetic growth in the available light.

Microplankton are microscopic organisms (fauna or flora, i.e., animal or plant) less than 63 μm (micrometers, microns) nominal size that have limited locomotion. By contrast, meioplankton are microscopic organisms (fauna or flora) greater than 63 μm to 500 μm nominal size that also have limited locomotion. The movement that occurs in both microplankton and meioplankton is dictated primarily by bulk water movement.

Microalgae is a type of microplankton flora which is defined as microscopic plants, including but not limited to single-cell and colonial algaes, that have a nominal size, singularly or aggregated, less than 63 μm in diameter. Meioalgae is microscopic plants, including by not limited to single-cell and colonial algaes, that have a nominal size, singularly or aggregated, between 63 μm and 500 μm. We will use the term small algae to refer to microalgae and large algae to refer to meioalgae.

Microplanktivorous culture is a marine aquatic system that facilitates and supports the reproduction and growth of organisms whose primary diet includes microplankton and meioplankton. Examples of microplanktivorous culture are organisms in the phyla Cnidaria or Ctenophora such as hard and soft corals, sea jellies, comb jellies; members of the order Clupeidae that include herring, sardines, menhaden, and anchovy; larval and early-life-stage shellfish before and immediately after settling such as oyster (e.g., Crassotrea virginica, Eastern Oyster) spat and seed, clams, mussels scallops, and the like or sedentary polychaete or bristle worms. While microplanktivorous may utilize all sizes of microalgae and meioalgae, the early life stages, spat and seed of oysters, clams, mussels, scallops, and like are unable to feed on meioalgae.

Target algae sizes range from <5 μm in nominal diameter such as smallest forms and early life stages of Chlorella spp., Isochrysis spp., Dunaliella spp., and the like. In order to provide an initial supply of the predetermined algae, breeding stock may be maintained in an algae genotype breeding stock container. This breeding stock provides the desired type of algae to start the culture if none are present in the nutrient supply to the culturing tank. Alternatively, for naturally occurring nutrient sources which may contain other, less desirable algae, the breeding stock container may introduce a sufficient amount of the desired algae in order to dominate the regeneration and growth process.

The requirement for utilizing natural water means that the culturing facility must be near a natural source of fresh or marine water containing microalgae thus limiting the location of the culturing facility or necessitating the transfer of the microalgae from its source to the culturing facility. If, alternatively, the microalgae is grown at the culturing facility without a means for selectively growing microalgae small enough, then a significant amount of nutrients may be wasted in growing the unneeded, oversize algae.

A nutrient source tank may be a container kept with aqueous nutrient media of sufficient high-concentration and appropriate nitrogen:phosphorus ratio to support algae growth by adding in relatively small amounts to the main culture system so as to not significantly dilute the bulk culture media. For example a 100 gallon bulk culture media should not have greater than a 10 gallon nutrient source addition so that the bulk culture media is not diluted more than 10%.

Algae nutrients comprising a nutrient medium include but are not limited to the macro nutrients of nitrogen in the forms of ammonia or nitrate, phosphorus in the form of phosphate, silicon in the form of silica; micronutrients as dissolved ions of iron, copper, molybdenum, zinc, magnesium, cobalt, manganese; and vitamins thiamine (B1), biotin (H), and cyanocobalamin (B12).

A chemistry control system may use dosing pumps to mix to nominal culture media concentrations nitrogen and phosphorus from concentrated stock solutions containing these singular nutrients, such as nitrate and phosphate. Dosing pumps are typically electromechanical pumps which can inject a specified amount of a chemical in response to a control signal.

An environment control system includes timing of photoperiod for artificial lights, sensing of submerged light levels to determine optimum light levels for the culture, and control the automatic harvest and subsequent dilution of culture biomass by fresh culture media in a continuous culture system. Further this environment control system may also measure the culturing medium temperature and control heating or cooling of the culture media to maintain an optimum culture temperature. It may also measure salinity and automatically add fresh water to the system to maintain a constant salinity that naturally increases through evaporation. Lastly the environment control system may monitor dissolved gas or outgas concentrations of carbon dioxide or oxygen to maintain optimal aeration of the culture media.

Effluent is usually biochemically treated by continuously circulating it through aerated tank containing quantities of aerobic and facultative bacteria. In one embodiment, an application would direct remaining effluent to an algae growth tank after a quantity of waste is removed from the effluent. Minerals such as nitrates and phosphates present in the waste effluent or liquor serve as nutrients for the growing algae.

There are practical limits of the nitrogen to phosphorus ratio (N:P) for the growth of algae. Phosphorus-limited algae growth is defined as a stoichiometric elemental (molar) nitrogen:phosphorus ratio greater than 30:1. Specific N:P ratios for target cultured algae species may range from 30:1 to >100:1 and depends on factors such as temperature or light. Specific N:P ratios promote the growth of specific genotypes of algae, thereby providing an environment for the culturing of algaes of a specific size, e.g., microalgae or meioalgae. Present embodiments are directed towards the selective culturing of microalgae maintained in ambient sub-tropical temperatures with natural light levels, potentially limited by high-density culture self-shading. Typical successful N:P ratios at these environmental conditions ranged from 200-250:1 N:P.

Certain values of the nitrogen to phosphorus ratio, N:P, promote the growth of specific genotypes of algae at the expense of other genotypes. Some genotypes, such as Chlorella spp. are small enough to be captured as food by shellfish seed that are less than 5 μm nominal diameter. These genotypes and early life stage algaes are typically filtered out of collected natural water used in aquaculture to protect equipment fouling and remove suspended clay sediments. Other genotypes or later life-stage algaes are too large to be available as food to the shellfish seed or larval stage planktivorous finfish (fry). Moreover, prepared algae food supplements focus on mature biomass with later life-stage, often clump cells together as part of the dewatering process and biomass concentration so that when the algae food media is reconstituted at lower concentrations the cells stick together forming larger nominal diameters.

For a shellfish seed culturing facility which does not desire to utilize unfiltered natural water sources, it may be more economical to maximize the production of small algae per unit of nitrogen and phosphorus since the goal is to maximize the spat production per unit nutrient addition and minimize the growth of non-useful algae. There may also be a savings in total energy consumption by utilizing artificial lighting to only grow microalgae and not waste it on growing non-useful algae.

Likewise, if it were desirable to only produce larger micro or meioalgae, the reverse process could be utilized where appropriate N:P ratios (approximately 30:1) that selectively promote the growth of larger micro and meioalgae genotypes.

The culture medium may be sterilized before being introduced to the algae culture. Sterilization may be performed, for example, by a sterilization apparatus which may utilize ultra-violet or gamma irradiation, ozone, chlorine, sodium hypochlorite, or other disinfection agent injection to insure maintenance of axenic algae culture. Axenic means relating to or denoting a culture that is free from living organisms other than the species required.

An example of an algae separation apparatus is disclosed in U.S. Pat. No. 9,321,057 titled Separation Apparatus and Process.

A sterilization apparatus is a process that kills all organisms entrained in the aquatic media as the culturing medium flows through the system. Typical methods include UV or gamma irradiation for relatively non-turbid systems, or ozone, chlorine, sodium hypochlorite (bleach) chemical injection in clear or turbid fluids.

A culturing water chemistry control system is a feedback control system that uses current automated or manual chemical analysis to determine the current state of the system and error from desired set point concentrations of control variables such as dissolved inorganic nitrogen, dissolved inorganic phosphorus, nitrogen-to-phosphorus ratio. Once the error is determined, a mathematical control algorithm determines the next addition of these chemicals by the nutrient dosing pumps to affect a change in the culturing water chemistry.

Example embodiments of the present invention may enable the preferential growth of specific algae genotypes from a culturing medium. Embodiments of the technology disclosed herein may be employed, for example, in the technical field of algae culturing. More particularly, the embodiments of the technology disclosed herein may be employed to grow microalgae with, for example, a 5 μm or less nominal diameter.

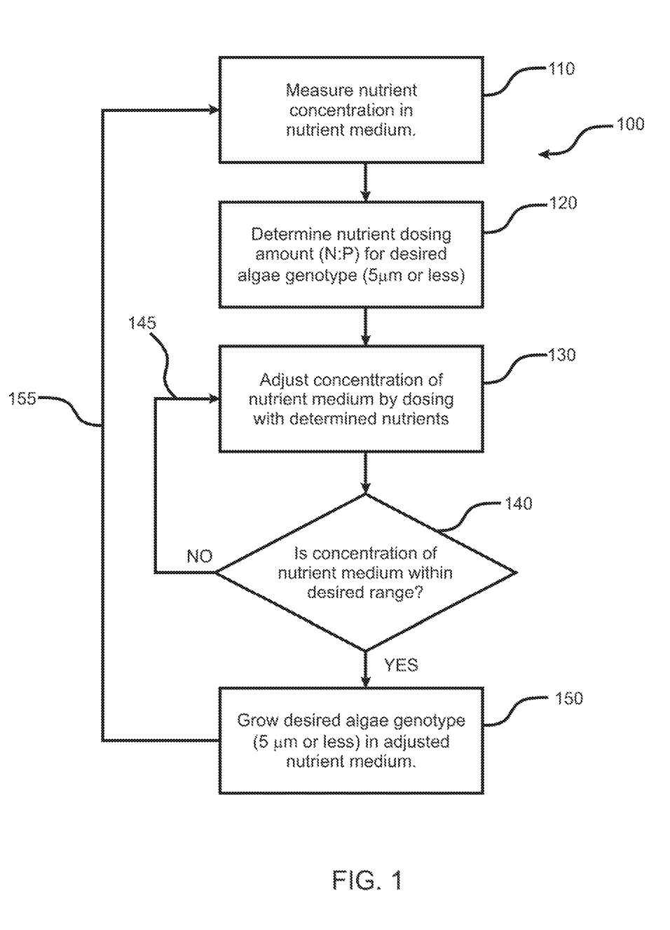

FIG. 1 is a process flow diagram depicting an example process as per an aspect of an embodiment of the present invention. As illustrated in this example 100, nutrient concentration of a nutrient medium may be measured at 110 to determine a nitrogen and phosphorous concentration of the nutrient medium. The nitrogen and phosphorous concentration can be measured using an ultra-sonic sensor, a near infrared sensor, an electrochemical probe, visible spectrophotometry, UV absorbance, or by other known analytical techniques such as chromatography. Ultrasonic sensors may be obtained from Sensotech.com. Near infrared sensors may be obtained from moistech.com. UV absorbance sensor may be obtained from Avantes.com, Visible spectrophotometry sensors may be obtained from vernier.com. Chromatography sensors may be found at Thermo Fisher Scientific. The sensors may be analog or digital and output signals that may be scaled based on a concentration calibration curve. The concentration measurements may be taken by placing a sensor inside a tank holding a nutrient medium. The measurements may also be taken outside a tank by taking one or more samples from the tank and measuring the sample(s) in a lab remote from the tank.

Based on the measured nitrogen and phosphorous concentration, a dosing amount of nitrogen and phosphorous may be determined at 120 in order to achieve a desired nitrogen to phosphorous ratio (N:P). For example, if the measured nitrogen to phosphorous ratio is measured to be 50:1 and the desired N:P ratio is 100:1, 50 additional parts of nitrogen may be determined to be added to the nutrient medium.

At 130, nitrogen and/or phosphorous may be added to the nutrient medium. The nitrogen and/or phosphorous may be added as a solid, liquid or gas. The nitrogen and phosphorous may be added by additions of sodium triphosphate and sodium nitrate. The nitrogen and/or phosphorous may be introduced into the nutrient medium by pumping, gravity feeding, screw conveying, or other known solid, liquid, or slurry material handling system.

The nitrogen and phosphorous concentration of the nutrient medium may be measured again at 140 to determine if additional dosing may be necessary to achieve the desired N:P ratio. If it is determined that the concentration of nutrient medium is not within a desired range at 140, the concentration may be adjusted again at 130 (by following path 145). Algae may be grown in the nutrient medium at 150 to achieve a desired algae genotype having, for example, a 5 μm or less nominal diameter. The process may be repeated (following path 155) in order to produce additional batches of the desired algae genotype.

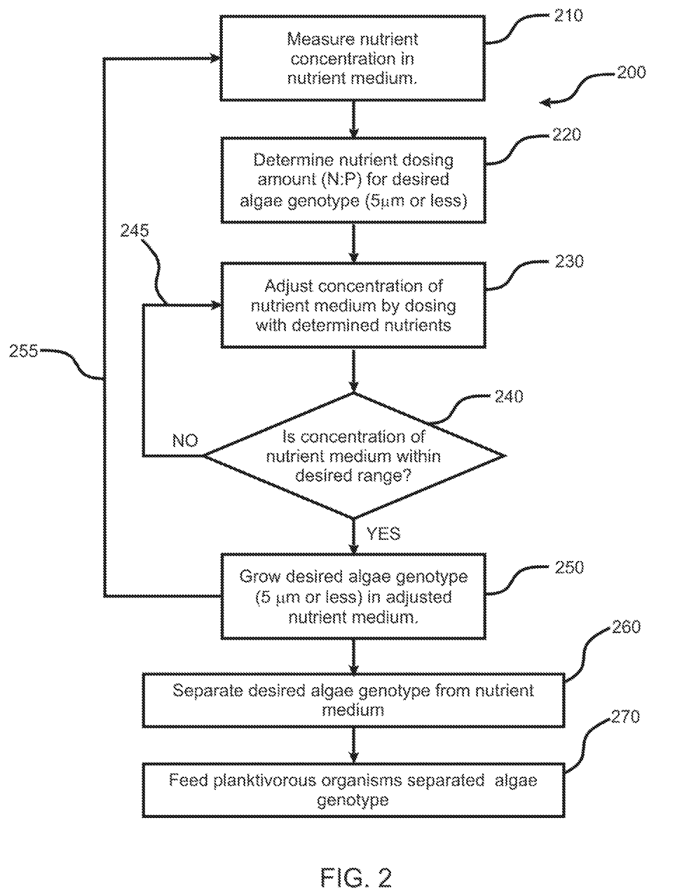

FIG. 2 is a process flow diagram depicting an example process as per an aspect of an embodiment of the present invention. As illustrated in this example 200, nutrient concentration of a nutrient medium may be measured at 210 to determine a nitrogen and phosphorous concentration of the nutrient medium.

Based on the measured nitrogen and phosphorous concentration, a dosing amount of nitrogen and phosphorous may be determined 220 in order to achieve a desired nitrogen to phosphorous ratio (N:P). At 230, the nitrogen and/or phosphorous may be added to the nutrient medium. The nitrogen and phosphorous concentration of the nutrient medium may be measured again at 240 to determine if additional dosing 245 may be necessary to achieve the desired N:P ratio.

Algae may be preferentially grown in the nutrient medium 250 to achieve a desired algae genotype having a 5 μm or less nominal diameter. The process may be repeated (following path 255) in order to produce additional batches of the desired algae genotype. The desired algae genotype may be separated from the nutrient medium at 260. The separated algae may be then fed to planktivorous organisms at 270.

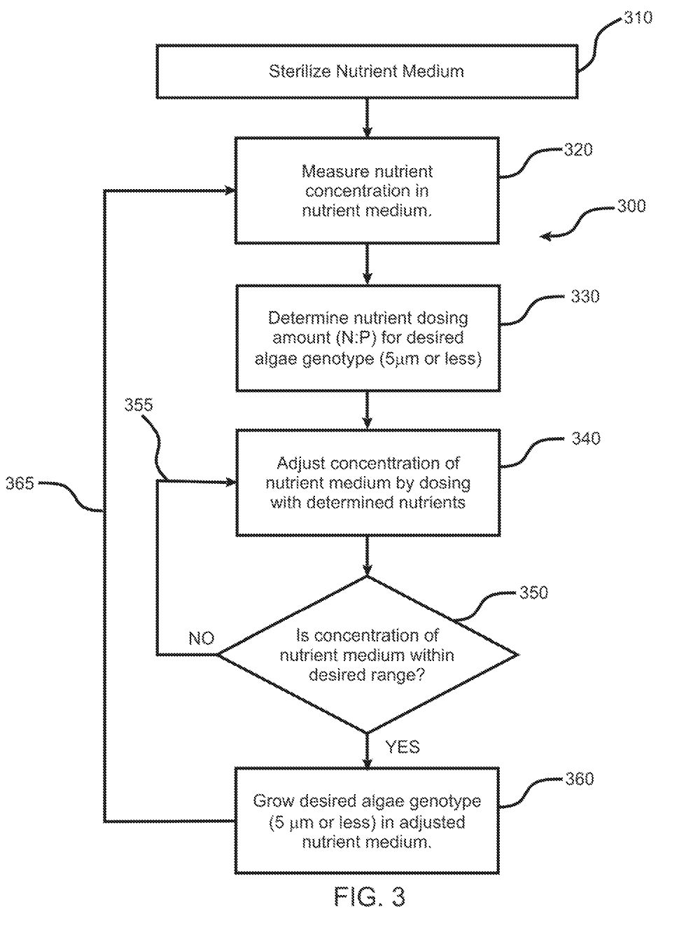

FIG. 3 is a process flow diagram depicting an example process as per an aspect of an embodiment of the present invention. As illustrated in this example 300, a nutrient medium may be sterilized at 310. The sterilization may occur by sterilizing at least one of the nutrient medium, the culture medium, and/or the separated microalgae. The sterilization may comprise a disinfecting agent additive system. The sterilization may comprise UV irradiation, gamma irradiation, ozone, chlorine, sodium hypochlorite, a combination thereof, and/or the like.

The nutrient concentration of the nutrient medium may be measured at 320 to determine a nitrogen and phosphorous concentration of the nutrient medium. Based on the measured nitrogen and phosphorous concentration, an appropriate dosing amount of nitrogen and phosphorous may be determined 330 in order to achieve a desired nitrogen to phosphorous ratio (N:P).

At 340, the nitrogen and/or phosphorous may be added to the nutrient medium. The nitrogen and phosphorous concentration of the nutrient medium may be measured again at 350 to determine if additional dosing 355 may be necessary to achieve the desired N:P ratio.

Algae may be grown in the nutrient medium 360 to achieve a desired algae genotype having a 5 μm or less nominal diameter. The process may be repeated 365 in order to produce additional batches of the desired algae genotype.

The nutrient source may be a source of water. The nutrient source may comprise wastewater industrial water, fresh water, marine water, brackish water, combinations thereof, and/or the like. According to an embodiment, a genotype of microalgae produced may have a nominal diameter of 5 μm or less. The nominal diameter of the microalgae genotype may be, for example, between 2 and 5 μm. The genotype may comprise Chlorella spp., Isochrysis spp., Dunaliella spp., and/or the like. According to an embodiment, the nitrogen to phosphorous ratio (N:P) may be greater than 100:1. According to an embodiment, N:P ratio may be, for example, approximately 200:1-250:1. According to an embodiment, “approximately” may refer to a ratio within 10% of the referenced value. According to another embodiment, “approximately” may account for a margin of error of a measurement probe. According to another embodiment, “approximately” may account for a margin of error of a measurement device.

The microalgae produced in an embodiment may be formed in a sterile and/or axenic manner. Axenic means relating to or denoting a culture that may be free from living organisms other than the species required. The sterilization may occur by sterilizing at least one of the nutrient medium, the culture medium, and/or the separated microalgae. The sterilization may comprise a disinfecting agent additive system. The disinfecting agent additive system may comprise a sterilization tank and pump that introduces a sterilizing medium such as chlorine, sodium hypochlorite, or ozone into the nutrient tank. The sterilization may comprise UV irradiation, gamma irradiation, ozone treatment, chlorine treatment, sodium hypochlorite treatment, a combination thereof, and/or the like.

The microalgae may be employed as a food for organisms. Suitable organisms may comprise planktivorous organisms. Examples of planktivorous organisms comprise hard coral, soft coral, sea jellies, comb jellies, herring, sardines, menhaden, anchovy, oyster, oyster spat and seed, clams, mussels, scallops, sedentary polychaete, bristle worms, and/or the like.

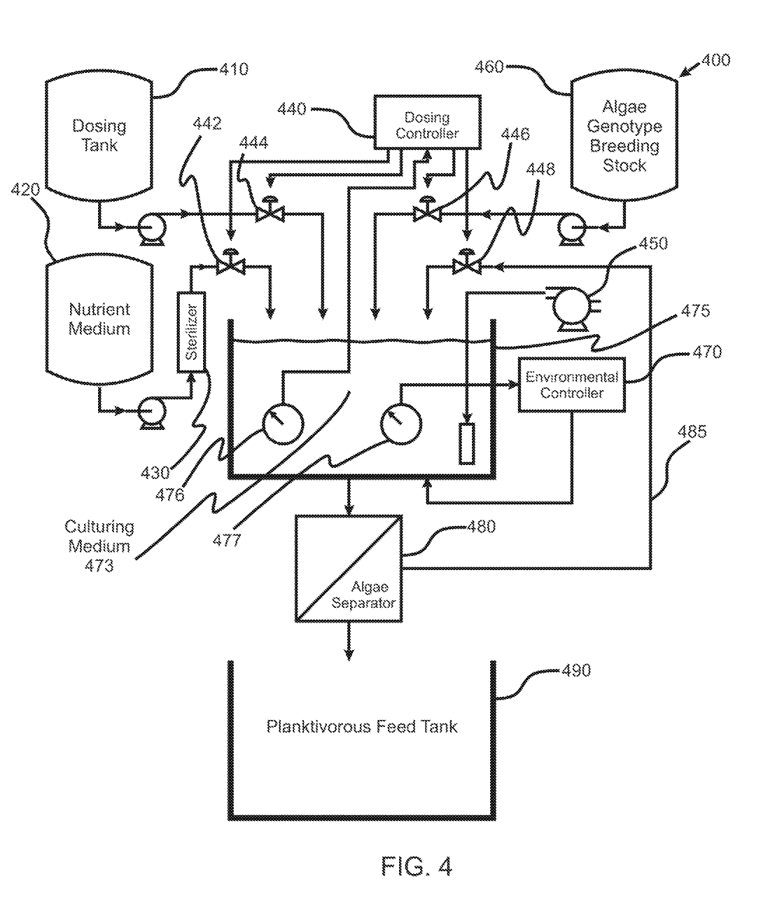

FIG. 4 is an example diagram depicting an example culturing apparatus as per an aspect of an embodiment. As illustrated in this example 400, the apparatus may comprise a culturing tank 475. The culturing tank 475 containing culturing medium 473 may be made of a material comprising plastics (for example, polyethylene, polypropylene), fiberglass, concrete, stone, or metal. The culturing tank may further be an earthen tank.

The culturing tank 475 may comprise a concentration sensor 476 configured to measure the nitrogen and phosphorous concentration of a liquid nutrient medium.

The culturing tank 475 may also comprise an environmental sensor 477 configured to measure environmental parameters within the culturing tank 475.

An aeration system 450 may be employed to aerate the culturing tank 475.

Nutrient medium tank 420 may hold the nutrient medium for transfer to a sterilization system 430. The sterilization system may be connected to an input into the culturing tank 475. A dosing controller 440 may be configured to receive input from the concentration sensor 476 and manipulate a control valve 444 connected to the sterilizer 430 based on the received input. This connection may facilitate transfer of the sterilized liquid nutrient source.

Dosing tank 410 may hold dosing nutrients for transfer the culturing tank 475. A dosing controller 440 may be configured to receive input from the concentration sensor 476 and manipulate a control valve connected to the dosing tank 410 and nutrient medium tank 420 based on the received input. The concentration sensor 476 may send data to the dosing controller 440. The dosing controller 440 may compare the received value with a programed target value and adjust the control valve in order to achieve a target value for dosing.

An algae genotype breeding stock tank 460 may be employed to hold a breeding stock for producing an algae genotype. A dosing controller 440 may be configured to receive input from the concentration sensor 476 and manipulate a control valve 446 connected to the algae genotype breeding stock tank 460 based on the received input. The breeding stock may be employed to preferentially grow a desired genotype of algae. The breeding stock may, for example, comprise Chlorella spp., lsochrysis spp., Dunaliella spp., and/or the like algae as a desired genotype. The breeding stock may be obtained from natural environment or may be isolated from the environment. The breeding stock may be an axenic algae culture. The axenic algae culture may be obtained from ncma.bigelow.org. The breeding stock may be added to a culturing medium at a concentration of 10⁴ -10⁵ algae per milliliter.

An environmental controller 470 may be configured to receive input from the environmental sensor 477 and to manipulate environmental conditions in response to the received input. The environmental controller 470 may receive input from the environmental sensor 477. The received input may be compared to a target value. The environmental controller 470 may adjust the environmental conditions to achieve the target value. For example, environmental controller 470 may manipulate the amount of light input, aeration intensity, temperature, pH, or salinity within the culturing tank 475. The light input (visible light, 400-700 nm) may be controlled at 10-1000 μmol m⁻² s⁻¹. The temperature may be controlled at 0-30° C. The pH may be controlled at 6.5-8.5. The salinity may be controlled at 0-40 ppt.

The dosing controller 440 or environmental controller 470 may comprise any combination of one or more of a proportional controller, integral controller, or derivative controller. The dosing controller 440 or environmental controller 470 may comprise a closed loop controller, feedback controller, adaptive controller, machine learning controller, optimal controller, and/or feed-forward controller, combinations thereof or the like. Optimal control deals with the problem of finding a control law for a given system such that a certain optimality criterion may be achieved. It is an extension of the calculus of variations, and is a mathematical optimization method for deriving control policies. Machine learning control (MLC) is a subfield of machine learning, intelligent control and control theory which solves optimal control problems with methods of machine learning. Machine learning may also be called artificial intelligence

The cultured microalgae produced in culturing tank 475 may be sent to an algae separator 480 configured to separate the microalgae from the nutrient source. The algae separator 480 may comprise a sedimentation system, membrane separation system, flow through centrifugation, plate and frame filtration, rotary filtration, parabolic screen filtration, and/or a flocculation system. The separation system may be the type discussed, for example, in U.S. Pat. No. 9,321,057, herein incorporated by reference.

The separated microalgae may be sent to a planktivorous feed tank 490 configured to hold planktivorous organisms. Recycled culture media 485 may be sent to the culturing tank 475 via a recycled culture mixing valve 448.

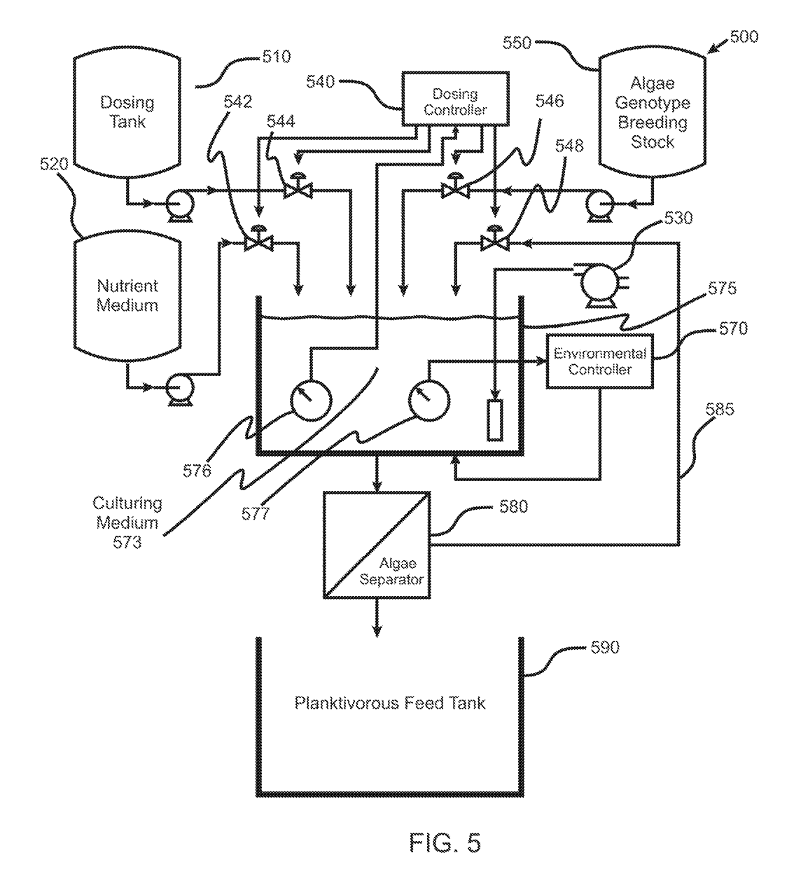

FIG. 5 is an example diagram depicting an example culturing apparatus as per an aspect of an embodiment of the present invention. As illustrated in this example 500, the apparatus may comprise a culturing tank 575 containing culturing medium 573. The culturing tank may comprise a concentration sensor 576 configured to measure the nitrogen and phosphorous concentration of a liquid nutrient medium. the sensor may performance analysis of the culturing medium. The culturing tanks also may comprise an environmental sensor 577 configured to measure the environmental parameters within the culturing tank 575. An aeration system 540l may be employed to aerate the culturing tank 575. A nutrient medium tank 520 holds the nutrient medium for transfer to the culturing tank 575. A dosing tank 510 holds dosing nutrients for transfer the culturing tank 575. A dosing controller 540l may be configured to receive input from the concentration sensor 576 and manipulate a control valve connected to the dosing tank 510 and nutrient medium tank 520 based on the received input. An algae genotype breeding stock tank 550 may be employed to hold a breeding stock for producing the desired algae genotype. An environmental controller 570 may be configured to receive input from the environmental sensor 577 may analyze the environment and manipulate environmental conditions in response to the received input. The cultured microalgae produced in culturing tank 575 may be sent to an algae separator 580 configured to separate the microalgae from the nutrient source. The separated microalgae may be sent to a planktivorous feed tank 590 configured to hold planktivorous organisms.

The dosing tank may not be limited to a single tank. The dosing system may include multiple tanks. The multiple tanks may each include independent control valves and transfer systems. The transfer systems in any embodiment of the invention may be not limited to pumps as shown in FIG. 4 and FIG. 5. The transfer system may include pressure, mechanical, air-lift, and gravity transfer systems.

The sterilization system 630 may not be limited in placement. The sterilization system may be configured to sterilize cultured algae. The sterilization system may be positioned in any arrangement of the culturing apparatus. The sterilization system may be arranged to sterilize the nutrient medium from the nutrient medium tank or the culture tank. The sterilization system may comprise a disinfecting agent additive system. The disinfecting agent additive system may be used to provide sodium hypochlorite at a concentration of 0.5-1.0 wt % to a nutrient medium. The sterilization system may comprise UV irradiation, gamma irradiation, autoclaving, ozone, chlorine, sodium hypochlorite, a combination thereof, and/or the like.

The dosing controller may be configured to operate a control valve connected to a dosing tank and the nutrient tank. The control valve may comprise a sluice gate valve, a needle control valve, or proportional control valve, or other known control valve suitable for controlling the amount of dosing into the nutrient tank. A sluice gate valve may allow for safe and reliable control of fluid flow. The needle control valve may be obtained from Omega or Brooks Instrument. The proportional control valve may be obtained from Parker Hydraulic and Pneumatic. The sluice valve may be obtained from The Valve Company. The concentration sensor may be an in situ analyzer. The concentration sensor may comprise ultrasonic sensors, near infrared sensors, and/or the like. Ultrasonic sensors may be obtained from Banner Engineering. Near infrared sensors may be obtained from Omega Engineering or Optris. The placement and arrangement of the sensors may not be limited and may be positioned inside or outside of the culture tank. The dosing and environmental controller may be integrated or independent. The environmental sensors may comprise at least one of a temperature sensor, a light sensor, a salinity sensor, dissolved gas sensor, and/or the like.

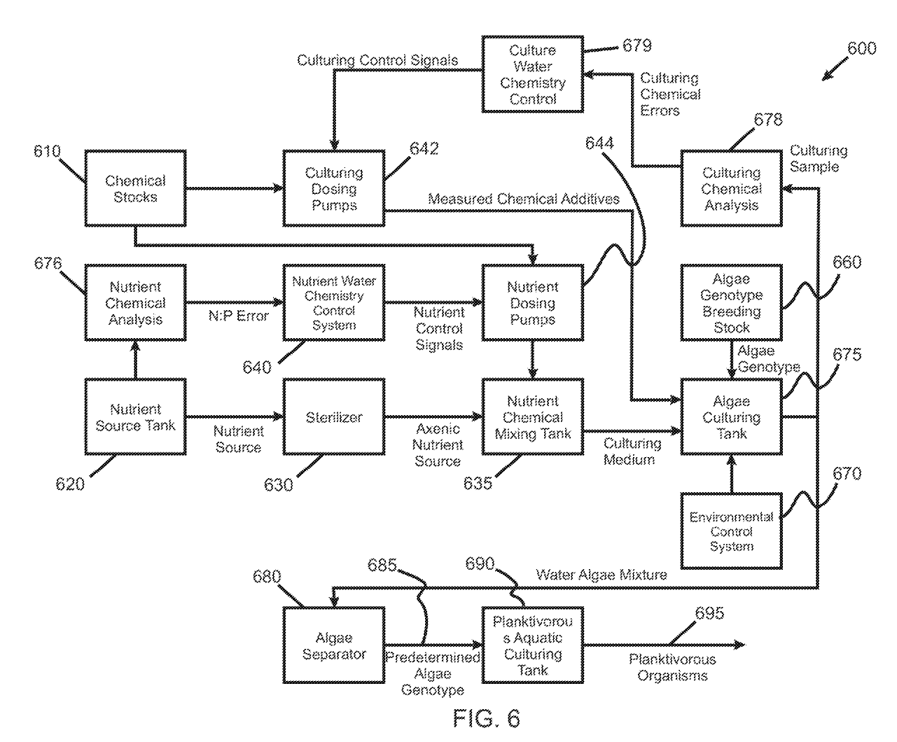

FIG. 6 is a process flow diagram depicting an example process as per an aspect of an embodiment of the present invention. As illustrated in this example 600, nutrient concentration of a nutrient medium in a nutrient source tank 620 may be measured at nutrient chemical analysis 676 to determine a nitrogen and phosphorous concentration of the nutrient medium. The concentration measurements may be taken by placing a sensor inside a tank holding a nutrient medium such as an algae culturing tank, 675. A nutrient chemistry control system 640 takes the N:P error and produces nutrient control signals which are applied to the nutrient dosing pumps, 644. The nutrient dosing pumps insert nutrients into the nutrient chemical mixing tank 635 to produce a culturing medium. This nutrient source may be sterilized 630. This culturing medium may be introduced into an algae culturing tank 675.

Based on the measured nitrogen and phosphorous concentration through the use of the culturing chemical analysis, 678, a dosing amount of nitrogen and phosphorous may be determined by the culture water chemistry control system at 679 in order to achieve a desired nitrogen to phosphorous ratio (N:P). The culturing water chemistry control system actuates the culture dosing pumps 642 to inject the correct amount of chemicals from chemical stocks, 610 to the algae culturing tank 675.

At the nutrient chemical mixing tank, 635, nitrogen and/or phosphorous may be added to the nutrient medium.

The nitrogen and phosphorous concentration of the nutrient medium may be measured again at 678 to determine if additional dosing may be necessary to achieve the desired N:P ratio. If it is determined that the concentration of nutrient medium is not within a desired range at 675, the concentration may be adjusted again by taking chemical stocks at 610 and passing them through culturing dosing pumps 642 to the algae culturing tank 675.

The cultured microalgae produced may be grown from breeding stocks 660 supplied to the algae culturing tank 675. An environmental control system 670 may be used to maintain physicochemical conditions within the algae culturing tank 675 to insure optimal growth of the cultured microalgae. The cultured microalgae produced in algae culturing tank 675 may be sent to an algae separator 680 configured to separate the microalgae from the culturing medium. The separated microalgae, the predetermined algae genotype 685, may be sent to a planktivorous aquatic culturing tank 690 configured to hold planktivorous organisms. The output of the planktivorous aquatic culturing tank 690 is the desired planktivorous organisms 695.

While various embodiments have been described above, it should be understood that they have been presented by way of example, and not limitation. It will be apparent to persons skilled in the relevant art(s) that various changes in form and detail may be made therein without departing from the spirit and scope. In fact, after reading the above description, it will be apparent to one skilled in the relevant art(s) how to implement alternative embodiments. Thus, the present embodiments should not be limited by any of the above-described exemplary embodiments.

Aspects of the present invention are disclosed in the foregoing description and related figures directed to specific embodiments of the invention. Those skilled in the art will recognize that alternate embodiments may be devised without departing from the spirit or the scope of the claims. In this specification, “a” and “an” and similar phrases are to be interpreted as “at least one” and “one or more.” References to “an” embodiment in this disclosure are not necessarily to the same embodiment.

In this specification, “a” and “an” and similar phrases are to be interpreted as “at least one” and “one or more.” References to “an” embodiment in this disclosure are not necessarily to the same embodiment.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

In addition, it should be understood that any figures that highlight any functionality and/or advantages, are presented for example purposes only. The disclosed architecture is sufficiently flexible and configurable, such that it may be utilized in ways other than those shown. For example, the steps listed in any flowchart may be re-ordered or only optionally used in some embodiments. Elements in the figures with the dashed lines may be indicative of optional elements that may be employed, in various combinations to create alternative embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112. Claims that do not expressly include the phrase “means for” or “step for” are not to be interpreted under 35 U.S.C. 112.

1. A method comprising:
measuring a nutrient source Nitrogen and Phosphorous concentration of a liquid nutrient source;
preparing a culturing medium with a ratio of Nitrogen to Phosphorous (N:P) that is greater than 100:1 by adjusting the nutrient source Nitrogen and Phosphorus concentration of the liquid nutrient source; and
culturing a microalgae in the culturing medium.

2. The method of claim 1, wherein the liquid nutrient source comprises at least one of:
wastewater;
rain water;
industrial water;
river water;
fresh water;
marine water; or
brackish water.

3. The method of claim 1, wherein the microalgae has a nominal diameter of less than 5 micrometers.

4. The method of claim 1, further comprising sterilizing the liquid nutrient source.

5. The method of claim 1, further comprising
measuring Nitrogen and Phosphorous concentration of the culturing medium; and
adjusting Nitrogen and Phosphorus concentration of the culturing medium so that the ratio of Nitrogen to Phosphorous (N:P) of the culturing medium is greater than 100:1.

6. A method comprising:
measuring Nitrogen and Phosphorous concentration of a liquid culturing medium;
adjusting Nitrogen and Phosphorus concentration of the liquid culturing medium to a ratio of Nitrogen to Phosphorous (N:P) that is greater than 100:1; and
culturing microalgae in the culturing medium.

7. The method of claim 6, wherein adjusting the culturing medium is done utilizing at least one of:
wastewater;
industrial water;
fresh water;
marine water; or
brackish water.

8. The method of claim 6, wherein adjusting the culturing medium is done utilizing chemical stocks.

9. The method of claim 6, wherein the microalgae has a nominal diameter of less than 5 micrometers.

10. The method of claim 6, wherein the ratio of N:P is approximately 200:1-250:1.

11. The method of claim 6, further comprising sterilizing the liquid nutrient source.

12. The method of claim 6, further comprising sterilizing the liquid nutrient source prior to adjusting the Nitrogen and Phosphorus concentration of the nutrient medium.

13. The method of claim 6, further comprising
measuring Nitrogen and Phosphorous concentration of the culturing medium; and
adjusting Nitrogen and Phosphorus concentration of the culturing medium so that the ratio of Nitrogen to Phosphorous (N:P) of the culturing medium is greater than 100:1.

14. An apparatus comprising:
a culturing tank;
a concentration analyzer configured to measure a Nitrogen and Phosphorous concentration of a culturing medium in a culturing tank; and
a dosing controller configured to adjust the Nitrogen and Phosphorous concentration of the culturing medium by the introduction of a nutrient medium into the culturing tank in response to measurements by the concentration analyzer of the culturing medium.

15. An apparatus according to claim 14 further comprising
an environmental sensor configured to measure environmental conditions in the culturing tank; and
an environmental controller configured to maintain a predetermined environmental condition within the culturing tank, by controlling environmental actuators, in response to measurements by the environmental sensor of the culturing medium.

16. The apparatus of claim 14, further comprising a sterilizing tank, wherein the sterilizing tank receives and sterilizes the nutrient medium from the nutrient medium tank.

17. The apparatus of claim 14, further comprising an aeration system connected to the culturing tank.

18. The apparatus of claim 14, further comprising an algae separator for separating algae from a culturing medium.

19. The apparatus of claim 14, wherein the environmental analyzer comprises at least one of:
a temperature sensor;
a light sensor;
a salinity sensor; and
a dissolved gas sensor.

20. The apparatus of claim 14, wherein the dosing controller comprises at least one of:
a proportional controller;
an integral controller; and
a derivative controller.
optimal controller;
adaptive controller;
machine learning controller;
feed-forward controller;
an integral controller; and
a derivative controller.
